(19) United States
(12) Patent Application Publication
(10) Pub. No.: US 2022/0202581 A1
(43) Pub. Date: Jun. 30, 2022

(54) TIBIAL COMPONENT

(71) Applicant: TEIJIN NAKASHIMA MEDICAL CO., LTD., Okayama-shi, Okayama (JP)

(72) Inventors: Dai KIYOTOMO, Okayama-shi (JP); Takayuki INOUE, Okayama-shi (JP)

(21) Appl. No.: 17/602,698

(22) PCT Filed: Feb. 18, 2020
(86) PCT No.: PCT/JP2020/006159

§ 371 Date: Oct. 8, 2021

(30) Foreign Application Priority Data
Apr. 11, 2019 (JP) 2019-075261

(51) Int. Cl. A61F 2/38; A61F 2/30

(52) U.S. Cl. CPC A61F 2/389; A61F 2/30749

(57) ABSTRACT

A tibial component for use in an artificial knee joint includes: a base fixed to an upper resection surface of a tibia, the base including an inner sliding surface and an outer sliding surface that are formed on an upper surface of the base, the inner sliding surface receiving a medial condyle of a femoral component, the outer sliding surface receiving a lateral condyle of the femoral component. The base includes a recess for avoiding interference between the base and a posterior cruciate ligament, the recess being formed in the base such that an opening of the recess faces backward. In a medial-lateral direction, a center of the recess is positioned inward relative to a center of the base.

### TECHNICAL FIELD

The present invention relates to a tibial component for use in an artificial knee joint.

### BACKGROUND ART

An artificial knee joint replaces a knee joint of a patient having, for example, knee osteoarthritis, chronic rheumatoid arthritis, osteoma, or an external wound. The artificial knee joint includes a femoral component and a tibial component. The femoral component substitutes for a part of a femur. The tibial component substitutes for a part of a tibia. In some cases, the artificial knee joint includes a patellar component.

In general, the femoral component includes a medial condyle and a lateral condyle, and the tibial component includes an inner sliding surface that receives the medial condyle and an outer sliding surface that receives the lateral condyle (see Patent Literature 1).

To be more specific, the tibial component includes a plate-shaped base that is fixed to an upper resection surface of the tibia. The inner sliding surface and the outer sliding surface are formed on the upper surface of the base.

### CITATION LIST

### Patent Literature

PTL 1: Japanese Patent No. 3781186

### SUMMARY OF INVENTION

### Technical Problem

In some cases, the proximal portion (femur-side end portion) of the tibia is resected planarly such that the upper resection surface, which receives the lower surface of the base of the tibial component, is formed as a flat surface. In other cases, the proximal portion (femur-side end portion) of the tibia is resected in such a manner that an enthesis at which the posterior cruciate ligament connects to the tibia is left in an island shape on the upper resection surface. In either case, it is necessary to avoid interference between the base of the tibial component and the posterior cruciate ligament or to avoid interference between the base of the tibial component and the enthesis at which the posterior cruciate ligament connects to the tibia. In order to do so, a recess whose opening faces backward is formed in the base.

In a conventional tibial component, the recess is formed at the center of the base in the medial-lateral direction. The “medial-lateral direction” herein refers to a direction in which the lowest point of the medial condyle and the lowest point of the lateral condyle of the femoral component in a knee extended position are arranged. However, in the case of the tibial component having such a shape, particularly in a case where the enthesis at which the posterior cruciate ligament connects to the tibia is left in an island shape on the upper resection surface, there is a risk of interference between the base of the tibial component and the posterior cruciate ligament enthesis.

In view of the above, an object of the present invention is to provide a tibial component that is usable with no problem regardless of how the proximal portion of the tibia is resected.

### Solution to Problem

In order to solve the above-described problems, the inventors of the present invention conducted diligent studies. As a result, the inventors of the present invention have found that the enthesis at which the posterior cruciate ligament connects to the tibia is not positioned at, but positioned inward relative to, the center of the proximal portion of the tibia in the medial-lateral direction. The present invention has been made from such a point of view.

Specifically, a tibial component of the present invention is a tibial component for use in an artificial knee joint, the tibial component including a base fixed to an upper resection surface of a tibia, the base including an inner sliding surface and an outer sliding surface that are formed on an upper surface of the base, the inner sliding surface receiving a medial condyle of a femoral component, the outer sliding surface receiving a lateral condyle of the femoral component. The base includes a recess for avoiding interference between the base and a posterior cruciate ligament or for avoiding interference between the base and an enthesis at which the posterior cruciate ligament connects to the tibia, the recess being formed in the base such that an opening of the recess faces backward. In a medial-lateral direction, a center of the recess is positioned inward relative to a center of the base.

According to the above configuration, the recess is positioned inward in the medial-lateral direction. Therefore, in a case where the proximal portion of the tibia is resected flat, the recess makes it possible to avoid interference between the posterior cruciate ligament and the base, whereas in a case where the proximal portion of the tibia is resected such that the enthesis at which the posterior cruciate ligament connects to the tibia is left in an island shape, the recess allows the posterior cruciate ligament enthesis to be positioned therein. Therefore, the tibial component is usable with no problem regardless of how the proximal portion of the tibia is resected.

For example, in the medial-lateral direction, a distance from the center of the base to the center of the recess may be 2 to 10% of a width of the base.

In the medial-lateral direction, the inner sliding surface may be positioned inward relative to the center of the recess, and the outer sliding surface may be positioned outward relative to the center of the recess. According to this configuration, in the medial-lateral direction, the relative positional relationship between the femur and the tibia of a patient to whom the artificial knee joint is attached is the same as the relative positional relationship between the femur and the tibia of a healthy person.

### Advantageous Effects of Invention

The present invention provides a tibial component that is usable with no problem regardless of how the proximal portion of the tibia is resected.

### BRIEF DESCRIPTION OF DRAWINGS

FIG. 1 is an exploded perspective view of an artificial knee joint including a tibial component according to one embodiment of the present invention.

FIG. 2 is a sectional view showing an attached state of the artificial knee joint of FIG. 1.

FIG. 3 is a sectional view taken along line of FIG. 2 (the illustration of a femur and a tibia is omitted).

FIG. 4 is a plan view of a tibial component.

FIG. 5 is a drawing for describing the center of a recess.

FIG. 6 is a plan view of the tibial component according to a variation.

FIG. 7 is a plan view of the tibial component according to another variation.

### DESCRIPTION OF EMBODIMENTS

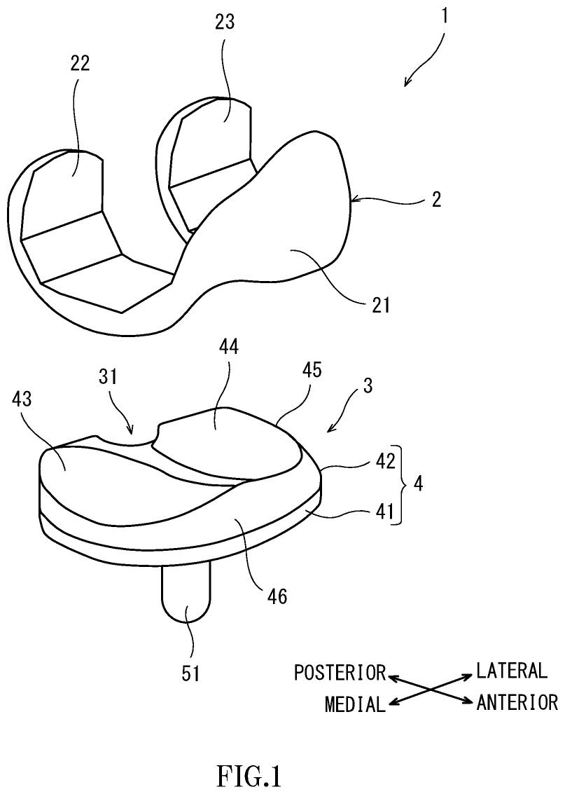

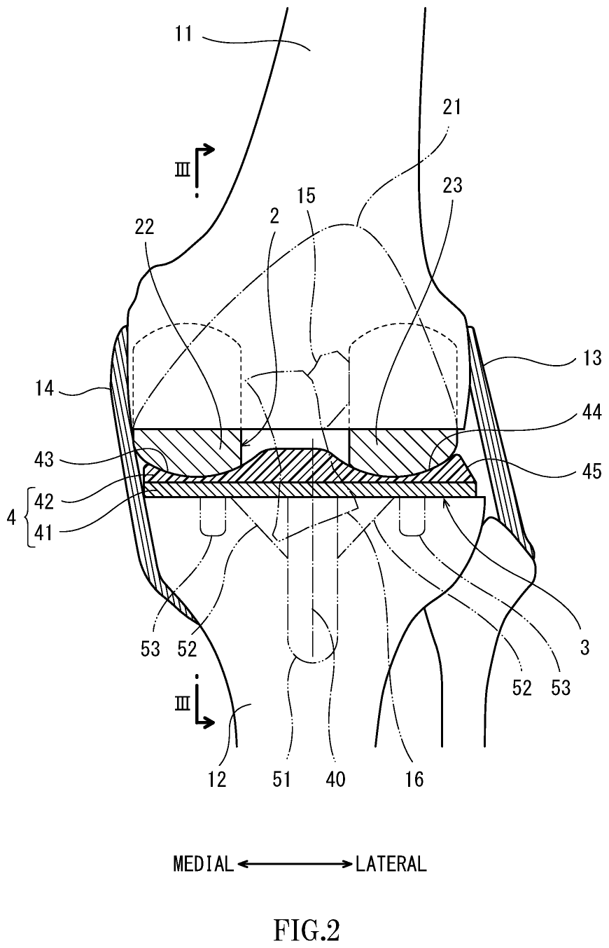

FIG. 1 shows an artificial knee joint 1 including a tibial component 3 according to one embodiment of the present invention. In the present embodiment, the artificial knee joint 1 is intended for a left leg. In FIG. 1, the direction toward the lower left is the medial direction; the direction toward the upper right is the lateral direction; the direction toward the lower right is the anterior direction; and the direction toward the upper left is the posterior direction. FIG. 2 is a sectional view showing an attached state of the artificial knee joint 1.

The artificial knee joint 1 includes a femoral component 2 in addition to the tibial component 3. Although not illustrated, the artificial knee joint 1 may include a patellar component. The femoral component 2 substitutes for a resected part of a femur 11. The tibial component 3 substitutes for a resected part of a tibia 12. It should be noted that, in FIG. 2, reference sign 13 indicates a lateral collateral ligament; reference sign 14 indicates a medial collateral ligament; reference sign 15 indicates an anterior cruciate ligament; and reference sign 16 indicates a posterior cruciate ligament.

The femoral component 2 is made of metal, such as a cobalt-chromium alloy or a titanium alloy. The femoral component 2 includes an anterior wall 21, a medial condyle 22, and a lateral condyle 23. The anterior wall 21 is fixed to an anterior resection surface of the femur 11. Each of the medial condyle 22 and the lateral condyle 23 extends from the lower end of the anterior wall 21 to the posterior side of the femur 11 in a manner to pass under the femur 11.

The medial condyle 22 and the lateral condyle 23 are spaced apart from each other. The gap between the medial condyle 22 and the lateral condyle 23 is intended for avoiding interference with the anterior cruciate ligament 15 and the posterior cruciate ligament 16. A patella groove, on which the patella or the patellar component slides, is formed in the lower portion of the anterior wall 21, such that the patella groove extends in the anterior-posterior direction that passes between the medial condyle 22 and the lateral condyle 23.

The external surface of the medial condyle 22 is a three-dimensional curved surface that is curved in the anterior-posterior direction and the medial-lateral direction. Similarly, the external surface of the lateral condyle 23 is also a three-dimensional curved surface that is curved in the anterior-posterior direction and the medial-lateral direction.

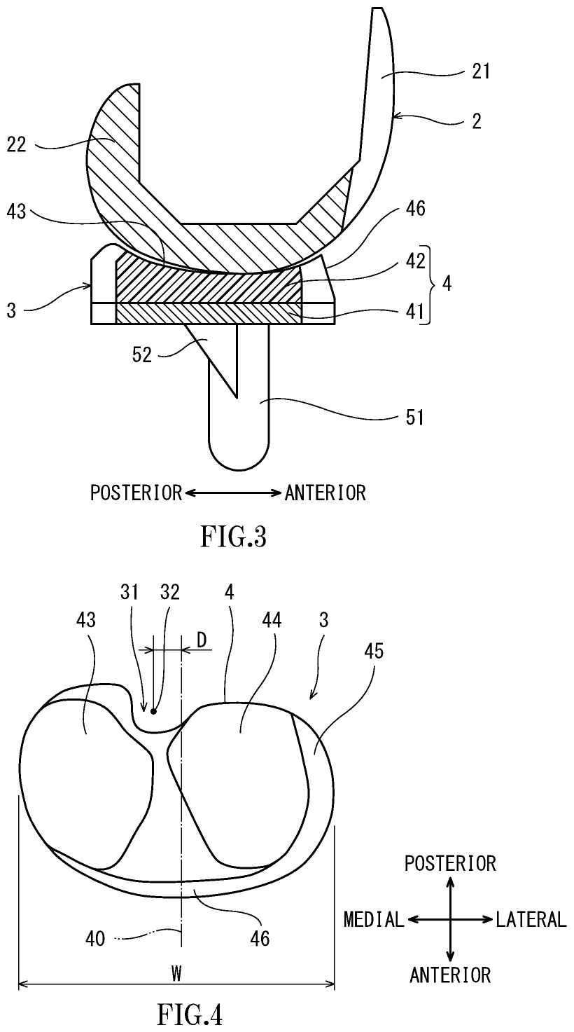

In the present embodiment, the tibial component 3 is of a type that is used in a case where the anterior cruciate ligament 15 is resected, but the posterior cruciate ligament 16 is preserved. Specifically, the tibial component 3 includes a plate-shaped base 4 and a stem 51. The base 4 is fixed to an upper resection surface of the tibia 12. The stem 51 extends downward from substantially the center of the base 4. As shown FIG. 2 and FIG. 3, the tibial component 3 further includes triangular keels 52, which are connected to the base 4 and the stem 51. The tibial component 3 may include, in addition to or instead of the stem 51 and the keels 52, a pair of pegs 53 (see FIG. 2) positioned below an inner sliding surface 43 and an outer sliding surface 44. The inner sliding surface 43 and the outer sliding surface 44 will be described below.

The base 4 includes a metal plate 41 and a resin plate 42, which are stacked together. The above-described stem 51 and keels 52 are integrated with the metal plate 41. The metal plate 41 is made of, for example, a cobalt-chromium alloy or a titanium alloy. The resin plate 42 is made of, for example, polyethylene.

The inner sliding surface 43, which receives the medial condyle 22 of the femoral component 2, and the outer sliding surface 44, which receives the lateral condyle 23 of the femoral component 2, are formed on the upper surface of the resin plate 42 (which is also the upper surface of the base 4).

The inner sliding surface 43 is a three-dimensional curved surface that is curved in the anterior-posterior direction and the medial-lateral direction. Similarly, the outer sliding surface 44 is also a three-dimensional curved surface that is curved in the anterior-posterior direction and the medial-lateral direction.

The base 4 further includes a recess 31 for avoiding interference between the base 4 and the posterior cruciate ligament 16 or for avoiding interference between the base 4 and an enthesis at which the posterior cruciate ligament 16 connects to the tibia 12. The recess 31 is formed in the base 4 such that the opening of the recess 31 faces backward. As shown in FIG. 4, in the medial-lateral direction, a center 32 of the recess 31 is positioned inward relative to a center 40 of the base 4. For example, in the medial-lateral direction, a distance D from the center 40 of the base 4 to the center 32 of the recess 31 is 2 to 10% of a width W of the base 4.

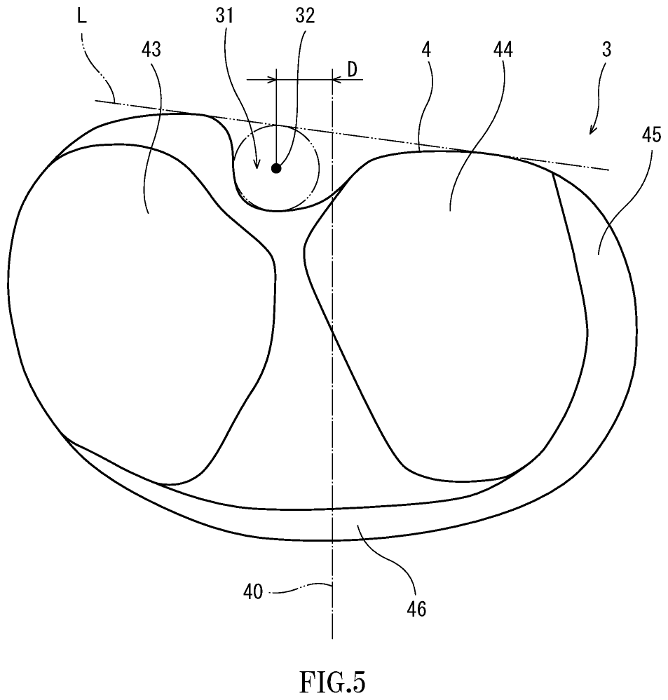

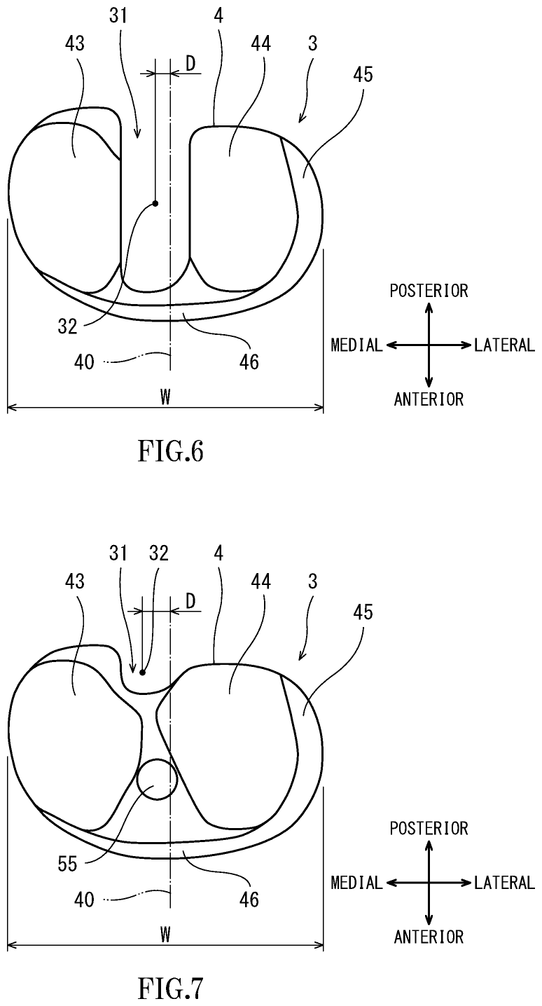

The “center 32 of the recess 31” herein refers to, as shown in FIG. 5, the center of a largest circle that is drawable in a region that is surrounded by the recess 31 and a tangent line L, which touches the base 4 from the posterior side at two points on both sides of the recess 31. In a case where a plurality of largest circles are drawable, for example, for the reason that the width of the recess 31 is constant (see FIG. 6, for example), or for the reason that the bottom of the recess 31 is parallel to the tangent line L, the center of the largest circle that is positioned in the middle among the plurality of largest circles (i.e., the middle point of a line that connects the center of the largest circle positioned on one end and the center of the largest circle positioned on the other end) is the center 32 of the recess 31.

Similar to the recess 31, the inner sliding surface 43 and the outer sliding surface 44 are positioned inward. In the present embodiment, in the medial-lateral direction, the inner sliding surface 43 is positioned inward relative to the center 32 of the recess 31, and the outer sliding surface 44 is positioned outward relative to the center 32 of the recess 31.

Further, as shown in FIG. 2 and FIG. 4, an inclined surface 45, which is inclined outward and downward, is formed on an end portion of the upper surface of the resin plate 42, the end portion protruding outward relative to the femoral component 2. In the present embodiment, the outer sliding surface 44 and the inclined surface 45 are adjacent to each other, and the boundary therebetween forms a ridge. The inclined surface 45 is intended for avoiding interference between the lateral collateral ligament 13 and the tibial component 3.

As shown in FIG. 3 and FIG. 4, also on the anterior end portion of the upper surface of the resin plate 42, an inclined surface 46 is formed, which is inclined outward and downward.

As described above, in the tibial component 3 of the present embodiment, the recess 31 is positioned inward in the medial-lateral direction. Therefore, in a case where the proximal portion of the tibia 12 is resected flat, the recess 31 makes it possible to avoid interference between the posterior cruciate ligament 16 and the base 4, whereas in a case where the proximal portion of the tibia 12 is resected such that the enthesis at which the posterior cruciate ligament 16 connects to the tibia 12 is left in an island shape, the recess 31 allows the posterior cruciate ligament enthesis to be positioned therein. Therefore, the tibial component 3 is usable with no problem regardless of how the proximal portion of the tibia 12 is resected.

(Variations)

The present invention is not limited to the above-described embodiment. Various modifications can be made without departing from the scope of the present invention.

For example, as shown in FIG. 6, the tibial component 3 may be of a type that is used in a case where both the anterior cruciate ligament 15 and the posterior cruciate ligament 16 are preserved. In this case, the depth of the recess 31 is deep, and the base 4 is substantially U-shaped. In the tibial component 3 shown in FIG. 6, the stem 51 and the keels 52 are eliminated.

Alternatively, as shown in FIG. 7, the tibial component 3 may be of a type that is used in a case where the posterior cruciate ligament 16 is preserved and the anterior cruciate ligament 15 is reconstructed. In this case, the base 4 is provided with a through-hole 55, which is positioned between the inner sliding surface 43 and the outer sliding surface 44. An artificial ligament or autologous tissue (e.g., collected patellar tendon tissue) that substitutes for the anterior cruciate ligament 15 is passed through the through-hole 55.

Further alternatively, although not illustrated, the tibial component 3 may be of a type that is used in a case where both the anterior cruciate ligament 15 and the posterior cruciate ligament 16 are resected. In this case, the tibial component 3 is provided with an upward projection that is positioned between the inner sliding surface 43 and the outer sliding surface 44.

It is not essential that the inner sliding surface 43 and the outer sliding surface 44 be positioned inward. Alternatively, the inner sliding surface 43 and the outer sliding surface 44 may be provided at bilateral symmetrical positions with respect to the center 40 of the base 4. However, in a case where the inner sliding surface 43 and the outer sliding surface 44 are positioned on both sides of the center 32 of the recess 31 as in the above-described embodiment, the relative positional relationship in the medial-lateral direction between the femur and the tibia of a patient to whom the artificial knee joint 1 is attached is the same as the relative positional relationship between the femur and the tibia of a healthy person.

### REFERENCE CHARACTERS LIST

1 artificial knee joint
2 femoral component
22 medial condyle
23 lateral condyle
3 tibial component
31 recess
32 center
4 base
40 center
43 inner sliding surface
44 outer sliding surface

1. A tibial component for use in an artificial knee joint, the tibial component comprising a base fixed to an upper resection surface of a tibia, the base including an inner sliding surface and an outer sliding surface that are formed on an upper surface of the base, the inner sliding surface receiving a medial condyle of a femoral component, the outer sliding surface receiving a lateral condyle of the femoral component, wherein
the base includes a recess for avoiding interference between the base and a posterior cruciate ligament or for avoiding interference between the base and an enthesis at which the posterior cruciate ligament connects to the tibia, the recess being formed in the base such that an opening of the recess faces backward, and
in a medial-lateral direction, a center of the recess is positioned inward relative to a center of the base.

2. The tibial component according to claim 1, wherein
in the medial-lateral direction, a distance from the center of the base to the center of the recess is 2 to 10% of a width of the base.

3. The tibial component according to claim 1, wherein
in the medial-lateral direction, the inner sliding surface is positioned inward relative to the center of the recess, and the outer sliding surface is positioned outward relative to the center of the recess.
